(19) United States
(12) Patent Application Publication
(10) Pub. No.: US 2022/0026357 A1
(43) Pub. Date: Jan. 27, 2022

(54) Portable Visible/near-infrared Spectrum Detection Device

(71) Applicant: Jiangsu University, Zhenjiang City (CN)

(72) Inventors: Qin Ouyang, Zhenjiang City (CN); Li Wang, Shuyang County (CN); Quansheng Chen, Zhenjiang City (CN); Zhiming Guo, Zhenjiang City (CN); Huanhuan Li, Zhenjiang City (CN); Jizhong Wu, Luoshan County (CN); Haihui Pan, Yancheng City (CN)

(21) Appl. No.: 17/329,780

(22) Filed: May 25, 2021

Related U.S. Application Data
(63) Continuation of application No. PCT/CN2021/072585, filed on Jan. 19, 2021.

(30) Foreign Application Priority Data
Jul. 24, 2020 (CN) CN202010720943.7

(51) Int. Cl. G01N 21/3563; G01N 21/359

(52) U.S. Cl. CPC G01N 21/3563; G01N 21/359

(57) ABSTRACT

The invention discloses a portable visible/near-infrared spectrum detection device, comprising a housing, a bottom plate, a screen support frame, a battery part on which a microprocessor is arranged, a detection part and a switch part, wherein the battery part, the detection part and the switch part are arranged in the housing; and the detection part comprises a spectrometer, a light source, a collimating mirror and the microprocessor. The device has the characteristics of rapidness, no damage and portability, and can realize rapid detection and early warning of food quality.

### CROSS REFERENCE TO RELATED APPLICATION

The invention relates to the technical field of food non-destructive detection, in particular to a portable visible/near-infrared spectrum detection device used for food quality evaluation and early warning.

### BACKGROUND OF THE INVENTION

With the rapid economic development, people's living standards have been continuously improved, and people are paying more and more attention to food quality and safety. In recent years, food quality problems have continuously emerged, which not only harm the rights and interests of consumers, but also disrupt the stable order of the food market. The traditional physical and chemical detection methods of food mainly include: high-performance liquid chromatography, gas chromatography, high-performance liquid chromatography-mass spectrometry, etc. Although the accuracy is high, the operation is complicated, the detection is time-consuming, the cost is high, and the operation of professional experimenters is required, and it is impossible to quickly feedback the quality information of the food.

Visible/near-infrared spectroscopy can characterize the quality characteristics of foods (meat, tea, fruits and vegetables, etc.) through characteristic spectra. It has the following characteristics: (1) the operation is simple, professionals are not needed, and cost of detection is low; (2) test samples hardly need pre-treatment, and chemical reagents are required to use; (3) multiple quality characteristics of samples can be detected at the same time; and (4) online detection is easy to achieve. Therefore, visible/near-infrared spectroscopy, as a fast, non-destructive and green analysis technique, has been widely used in the field of rapid food detection in recent years.

The desktop near-infrared spectrum detector has a complex structure, is expensive, has a large volume, and is not suitable for on-site online inspection of food. With the rapid development of science and technology, spectrometers, halogen lamps, etc. have developed in a stable and portable direction. After searching related patents, the patent “Portable Near-Infrared Spectrometer Solid Sample Detection Device with a Patent No. 201621106011.9 ” is found, the spectrometer to be fixed under a sample cup is used to detect solid samples in the sample cup, only solid samples can be detected, the shape of the detected samples is single, and a bracket, a base and a bolt are assembled and matched as support for sample detection, so that operation is complex, manual operation requirements in a detection process are high and deviation appears in operation to affect detection precision, and thus, application to the food field is relatively difficult, and an actual application range is limited. It is necessary to design a portable visible/near-infrared spectrum detection device to solve the problems existing in the prior art.

### SUMMARY OF THE INVENTION

The objective of the invention is to provide a portable visible/near-infrared spectrum detection device which solves the problems in the prior art, and realizes the detection purpose of higher detection precision, a low price, a smaller size and higher analysis speed.

To achieve the objective, the invention provides the following scheme: the invention provides a portable visible/near-infrared spectrum detection device which includes a housing, a bottom plate, a screen supporting frame, a battery part, a detection part and a switch part;

the bottom end and the top end of the housing are fixedly connected to the bottom plate and the screen supporting frame respectively, the battery part and the detection part are arranged in the housing; the switch part is arranged on the inner wall of the top surface of the housing; the battery part is electrically connected to the switch part; the switch part is electrically connected to the detection part;

the detection part includes a spectrometer, a light source, a collimating mirror and a microprocessor, wherein the spectrometer is arranged on the bottom plate, the collimating mirror is arranged on the left side of the spectrometer, the light source is arranged above the collimating mirror, a light source interface is formed in the side surface, close to the light source, of the housing; a spectrometer interface is formed below the light source interface; the spectrometer interface, the collimating mirror and the spectrometer are electrically connected; the light source interface is electrically connected to the light source, and the light source is electrically connected to the collimating mirror; a microprocessor is arranged above the battery part; the microprocessor is electrically connected to the spectrometer; an integrating sphere module, an optical fiber module and a transmission module are externally connected to the spectrometer interface;

an embedded type flat plate which is electrically connected to the switch part and the microprocessor separately is detachably connected on the screen supporting frame.

Preferably, two through holes are formed in the left side surface of the housing, and the spectrometer interface and the light source interface are separately embedded into the two through holes.

Preferably, the battery part includes a battery bin and a storage battery, where the battery bin is fixedly connected on the bottom plate, and the storage battery is positioned in the battery bin.

Preferably, the switch part includes a power supply switch, a system switch and a light source switch, where a strip-shaped hole and three through holes are formed in the screen supporting frame; the power supply switch, the system switch and the light source switch are detachably connected in three through holes in sequence; and an electric quantity display screen, which is connected to the storage battery electrically, is connected in the strip-shaped hole in an inserting mode.

Preferably, the storage battery is electrically connected to the power supply switch, the system switch and the light source switch separately; the power supply switch is electrically connected to the embedded type flat plate; the system switch is electrically connected to the spectrometer and the microprocessor, and the light source switch is electrically connected to the light source.

Preferably, the bottom surface of the spectrometer is fixedly connected to a spectrometer fixing frame which is fixedly connected onto the bottom plate.

Preferably, a heat dissipation hole is formed in the rear side surface of the housing, and a charging interface is arranged at one side, away from the light source interface, of the housing.

Preferably, the light source is a halogen lamp with a measuring range of 360-2400 nm.

Preferably, the spectrometer has a wavelength range of 345-1032 nm, a signal-to-noise ratio of 300:1, and a spectral resolution of 0.15-0.22 nm.

Preferably, a screen adapter plate is arranged on the screen supporting frame in an inclined mode, and the two side surfaces of the housing are fixedly connected to handles.

The invention discloses the following technical effects:

A housing, a spectrometer interface, a light source interface, a light source and a spectrometer are arranged, the spectrometer interface is connected to the spectrometer, the light source interface is connected to the light source, and the spectrometer interface is further connected to an integrating sphere module, an optical fiber module and a transmission module, so that three different acquisition ways are coupled for being suitable for spectrum collection and quality detection of food materials of different forms, a detection range is wide and practicability is strong.

The device further has a wireless network transmission function for transmitting food quality information to a food supervision platform, thereby realizing real-time monitoring of food quality, and being beneficial to food market supervision. The device can be powered by batteries, is more portable, and is suitable for food quality detection in different environments and different places.

### BRIEF DESCRIPTION OF THE FIGURES

In order to explain the embodiments of the invention or the technical solutions in the prior art more clearly, the following will briefly introduce the drawings that need to be used in the embodiments. Obviously, the drawings in the following description are only some embodiments of the invention. For those of ordinary skill in the art, without creative labor, other drawings can be obtained from these drawings.

FIG. 1 is a schematic structural diagram of the portable visible/near-infrared spectrum detection device of the invention.

FIG. 2 is a top view of FIG. 1.

FIG. 3 is an axonometric drawing of the housing of the invention.

FIG. 4 is an axonometric drawing of the screen supporting frame of the invention.

FIG. 5 is an axonometric drawing of the bottom plate of the invention.

FIG. 6 is an axonometric drawing of the battery bin of the invention.

FIG. 7 is an axonometric drawing of the spectrometer of the invention.

where, 1. housing; 1.1. heat dissipation hole; 1.2. spectrometer interface; 1.3. light source interface; 1.4. charging interface; 2. screen supporting frame; 3. embedded type flat plate; 4. screen adapter plate; 5. power supply switch; 6. system switch; 7. light source switch; 8. electric quantity display screen; 9. bottom plate; 10. light source, 11. spectrometer fixing frame; 12. spectrometer; 13. collimating mirror; 14. battery bin; 15. storage battery; 16. microprocessor; and 17. handle.

### DETAILED DESCRIPTION OF THE PREFERRED EMBODIMENTS

The technical solutions in the embodiments of the invention will be clearly and completely described below in conjunction with the accompanying drawings in the embodiments of the invention. Obviously, the described embodiments are only a part of the embodiments of the invention, rather than all the embodiments. Based on the embodiments of the invention, all other embodiments obtained by those of ordinary skill in the art without creative work shall fall within the protection scope of the invention.

In order to make the above objectives, features and advantages of the invention more obvious and understandable, the invention will be further described in detail below with reference to the accompanying drawings and specific embodiments.

### Embodiment I

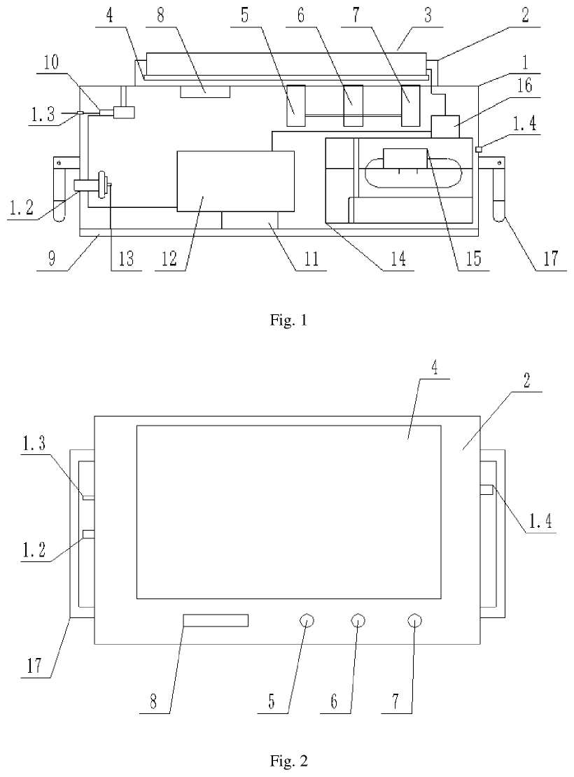

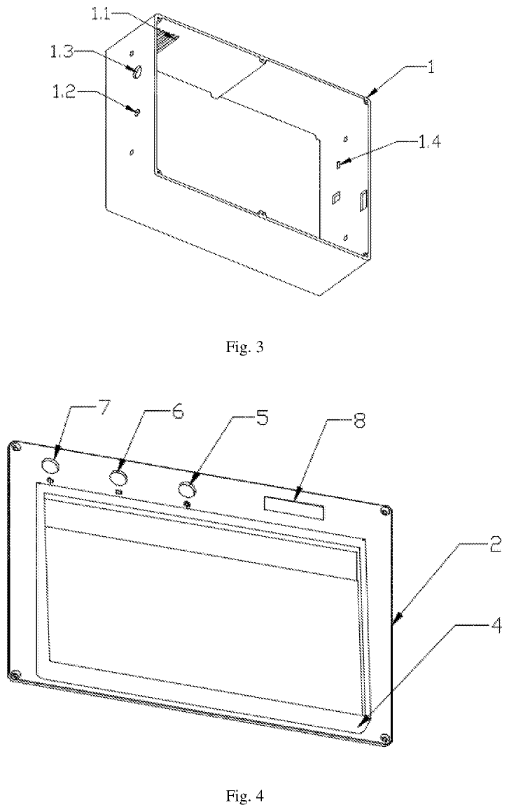

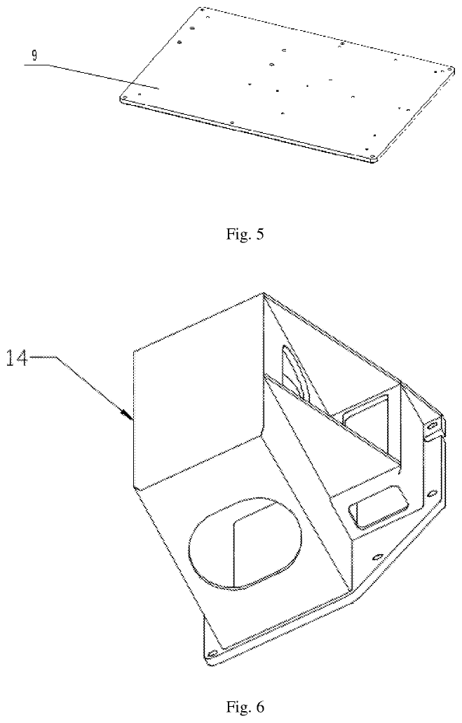

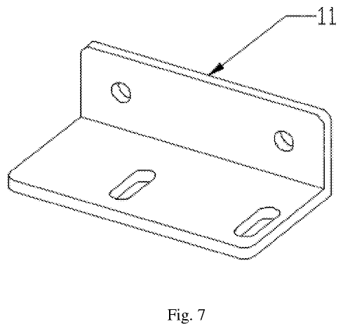

Refer to FIG. 1 to FIG. 7, the invention provides a portable visible/near-infrared spectrum detection device which includes a housing 1, a bottom plate 9, a screen supporting frame 2, a battery part, a detection part and a switch part; the bottom end and the top end of the housing 1 are fixedly connected to the bottom plate 9 and the screen supporting frame 2 respectively, the battery part and the detection part are arranged in the housing 1; the switch part is arranged on the inner wall of the top surface of the housing 1; the battery part is electrically connected to the switch part; and the switch part is electrically connected to the detection part;

the detection part comprises a spectrometer 12, a light source 10, a collimating mirror 13 and a microprocessor 16, wherein the spectrometer 12 is arranged on the bottom plate 9, the collimating mirror 13 is arranged on the left side of the spectrometer 12, the light source 10 is arranged above the collimating mirror 13, a light source interface 1.3 is formed in the side surface, close to the light source 10, of the housing 1; a spectrometer interface 1.2 is formed below the light source interface 1.3; the spectrometer interface 1.2, the collimating mirror 13 and the spectrometer 12 are electrically connected; the light source interface 1.3 is electrically connected to the light source 10, and the light source 10 is electrically connected to the collimating mirror 13; a microprocessor 16 is arranged above the battery part; the microprocessor 16 is electrically connected to the spectrometer 12; and an integrating sphere module, an optical fiber module and a transmission module are externally connected to the spectrometer interface 1.2;

an embedded type flat plate 3 which is electrically connected to the switch part and the microprocessor 16 separately is detachably connected on the screen supporting frame 2.

In a further preferred solution, two through holes are formed in the left side surface of the housing, and the spectrometer interface 1.2 and the light source interface 1.3 are separately embedded into the two through holes.

In a further preferred solution, the battery part includes a battery bin 14 and a storage battery 15, where the battery bin 14 is fixedly connected on the bottom plate 9, and the storage battery 15 is positioned in the battery bin 14.

In a further preferred solution, the switch part includes a power supply switch 5, a system switch 6 and a light source switch 7, where a strip-shaped hole and three through holes are formed in the screen supporting frame 2; the power supply switch 5, the system switch 6 and the light source switch 7 are detachably connected in three through holes in sequence; and an electric quantity display screen 8, which is connected to the storage battery 15 electrically, is connected in the strip-shaped hole in an inserting mode.

In a further preferred solution, the storage battery 15 is electrically connected to the power supply switch 5, the system switch 6 and the light source switch 7 separately; the power supply switch 5 is electrically connected to the embedded type flat plate 3; the system switch 6 is electrically connected to the spectrometer 12 and the microprocessor 16, and the light source switch 7 is electrically connected to the light source 10.

In a further preferred solution, the bottom surface of the spectrometer is fixedly connected to a spectrometer fixing frame 11 which is fixedly connected onto the bottom plate 9.

In a further preferred solution, a heat dissipation hole 1.1 is formed in the rear side surface of the housing 1, and a charging interface 1.4 is arranged at one side, away from the light source interface 1.3, of the housing 1.

In a further preferred solution, the light source 10 is a halogen lamp with a measuring range of 360-2400 nm, and is equipped with an integrated fan for keeping the light source cool and stable.

In a further preferred solution, the spectrometer 12 has a wavelength range of 345-1032 nm capable of covering the visible/near-infrared spectral region, a signal-to-noise ratio of 300:1, and a spectral resolution of 0.15-0.22 nm to achieve good scanned spectrum information, adopts a linear array silicon-based detector with a spectral wavelength range covering visible and short near-infrared spectrum wavebands, is low in price, is high in detection precision, and can effectively detect food quality characteristics and the high signal-to-noise ratio increases stability and flexibility of spectral data, and the high resolution provides abundant spectral information.

In a further preferred solution, a screen adapter plate 4 is arranged on the screen supporting frame 2 in an inclined mode, and the two side surfaces of the housing 1 are fixedly connected to handles 17.

The working principle of the portable visible/near-infrared detection device is as follows:

The integrating sphere or optical fiber is connected to the spectrometer interface 1.2, and the other end of the integrating sphere or optical fiber is connected to the light source interface 1.3; the power switch 5, system switch 6 and light source switch 7 are started simultaneously, so that the storage battery 15 and the embedded type flat plate 3 can be started; power is supplied to the spectrometer 12, the microprocessor 16 and the light source 10 through the storage battery 15; the collimating mirror 13 is electrically connected to the spectrometer 12 through an optical fiber, the light source interface 1.3 is electrically connected to the light source 10 through an optical fiber, the light source 10 and the collimating mirror 13 are electrically connected through an optical fiber, the microprocessor 16 is arranged above the battery part, the microprocessor 16 and the spectrometer 12 are electrically connected through a cable, and the embedded type flat plate 3 is electrically connected to the microprocessor 16 and the storage battery 15 through cables, and therefore, by connecting the integrating sphere or directly between the light source interface 1.3 and the spectrometer interface 1.2, the food can be detected through the integrating sphere. The visible/near-infrared light of food forms a loop among the light source 10, the collimating lens 13, the spectrometer 12 and the integrating sphere, that is, the spectral signal is collected by the collimating lens 13, and then transmitted to the spectrometer 12, processed by the microprocessor 16, and transmitted to the embedded type flat plate 3, the wavelength range of the spectrometer 12 is 345-1032 nm, which can effectively cover the visible/near-infrared spectrum; the signal-to-noise ratio of the spectrometer 12 is 300:1, the spectral resolution is 0.15-0.22 nm, the scanned spectrum information is good, the light source 10 is a halogen lamp with a measurement range of 360-2400 nm, equipped with an integrated fan, which can keep the light source 10 cool and stable.

### Embodiment II

The difference between this Embodiment and the Embodiment I is that the device of this Embodiment is equipped with a wireless network transmission function. According to the developed software system, it realizes spectral data collection, spectral data transmission and food quality detection result feedback, where the spectral data and the detection results are transmitted to the cloud end through the wireless function, and enterprises and regulatory agencies can monitor food quality in real time on a regulatory platform. In addition, in this Embodiment, a transmission part is connected between the light source 10 and the spectrometer 12, and a light intensity attenuator is additionally arranged between the light source 10 and the transmission part, which is more conducive to the accuracy of detection.

In the description of the invention, it is to be understood that the orientation or position relationship indicated by the terms “longitudinal”, “transverse”, “upper”, “lower”, “front”, “rear”, “left”, “right”, “vertical”, “horizontal”, “top”, “bottom”, “inside”, “outside”, etc. is based on the orientation or position relationship shown in the drawings, and is merely for convenience of description of the invention. It is not intended or implied that the device or component that is referred to has a particular orientation, is constructed and operated in a particular orientation, and thus is not to be construed as limiting the invention.

The above-mentioned embodiments only describe the preferred mode of the invention, and do not limit the scope of the invention. Without departing from the design spirit of the invention, variations and improvements, made to the technical solutions of the invention by those of ordinary skill in the art shall fall within the protection scope determined by the claims of the invention.

What is claimed is:

1. A portable visible/near-infrared spectrum detection device, comprising a housing (1), a bottom plate (9), a screen supporting frame (2), a battery part, a detection part and a switch part;
wherein the bottom end and the top end of the housing (1) are fixedly connected to the bottom plate (9) and the screen supporting frame (2) respectively, the battery part and the detection part are arranged in the housing (1); the switch part is arranged on the inner wall of the top surface of the housing (1); the battery part is electrically connected to the switch part; and the switch part is electrically connected to the detection part;
the detection part comprises a spectrometer (12), a light source (10), a collimating mirror (13) and a microprocessor (16), wherein the spectrometer (12) is arranged on the bottom plate (9), the collimating mirror (13) is arranged on the left side of the spectrometer (12), the light source (10) is arranged above the collimating mirror (13), a light source interface (1.3) is formed in the side surface, close to the light source (10), of the housing (1); a spectrometer interface (1.2) is formed below the light source interface (1.3); the spectrometer interface (1.2), the collimating mirror (13) and the spectrometer (12) are electrically connected; the light source interface (1.3) is electrically connected to the light source (10), and the light source (10) is electrically connected to the collimating mirror (13); a microprocessor (16) is arranged above the battery part; the microprocessor (16) is electrically connected to the spectrometer (12); and an integrating sphere module, an optical fiber module and a transmission module are externally connected to the spectrometer interface (1.2);
and an embedded type flat plate (3) which is electrically connected to the switch part and the microprocessor (16) separately is detachably connected on the screen supporting frame (2).

2. The portable visible/near-infrared spectrum detection device according to claim 1, wherein two through holes are formed in the left side surface of the housing, and the spectrometer interface (1.2) and the light source interface (1.3) are separately embedded into the two through holes.

3. The portable visible/near-infrared spectrum detection device according to claim 1, wherein the battery part comprises a battery bin (14) and a storage battery (15), the battery bin (14) is fixedly connected on the bottom plate (9), and the storage battery (15) is positioned in the battery bin (14).

4. The portable visible/near-infrared spectrum detection device according to claim 3, wherein the switch part comprises a power supply switch (5), a system switch (6) and a light source switch (7), a strip-shaped hole and three through holes are formed in the screen supporting frame (2); the power supply switch (5), the system switch (6) and the light source switch (7) are detachably connected in three through holes in sequence; and an electric quantity display screen (8), which is connected to the storage battery (15) electrically, is connected in the strip-shaped hole in an inserting mode.

5. The portable visible/near-infrared spectrum detection device according to claim 4, wherein the storage battery (15) is electrically connected to the power supply switch (5), the system switch (6) and the light source switch (7) separately; the power supply switch (5) is electrically connected to the embedded type flat plate (3); the system switch (6) is electrically connected to the spectrometer (12) and the microprocessor (16), and the light source switch (7) is electrically connected to the light source (10).

6. The portable visible/near-infrared spectrum detection device according to claim 1, wherein the bottom surface of the spectrometer (12) is fixedly connected to a spectrometer fixing frame (11) which is fixedly connected onto the bottom plate (9).

7. The portable visible/near-infrared spectrum detection device according to claim 1, wherein a heat dissipation hole (1.1) is formed in the rear side surface of the housing (1), and a charging interface (1.4) is arranged at one side, away from the light source interface (1.3), of the housing (1).

8. The portable visible/near-infrared spectrum detection device according to claim 1, wherein the light source (10) is a halogen lamp with a measuring range of 360-2400 nm.

9. The portable visible/near-infrared spectrum detection device according to claim 1, wherein the spectrometer (12) has a wavelength range of 345-1032 nm, a signal-to-noise ratio of 300:1, and a spectral resolution of 0.15-0.22 nm.

10. The portable visible/near-infrared spectrum detection device according to claim 1, wherein a screen adapter plate (4) is arranged on the screen supporting frame (2) in an inclined mode, and the two side surfaces of the housing (1) are fixedly connected to handles (17).
